(19) United States
(12) Patent Application Publication
(10) Pub. No.: US 2023/0310158 A1
(43) Pub. Date: Oct. 5, 2023

(54) Balloon-Tipped Pad Delivery Catheter

(71) Applicant: Tendyne Holdings, Inc., St. Paul, MN (US)

(72) Inventor: Preston James Huddleston, Maplewood, MN (US)

(73) Assignee: Tendyne Holdings, Inc., St. Paul, MN (US)

(21) Appl. No.: 18/061,618

(22) Filed: Dec. 5, 2022

Related U.S. Application Data
(60) Provisional application No. 63/325,821, filed on Mar. 31, 2022.

(51) Int. Cl. A61F 2/24; A61M 25/10

(52) U.S. Cl. CPC A61F 2/2487; A61M 25/10181; A61F 2220/0075

(57) ABSTRACT

A prosthetic heart valve delivery system includes a prosthetic heart valve, an anchor, and a catheter extending from a proximal end to a distal end configured to receive the prosthetic heart valve and the anchor. The catheter includes a balloon circumferentially surrounding the catheter at the distal end of the catheter, and an inflation lumen positioned radially outward of the catheter extending from the proximal end to the distal end, the inflation lumen in communication with the balloon.

### CROSS-REFERENCE TO RELATED APPLICATIONS

This application claims priority to the filing date of U.S. Provisional Pat. Application No. 63/325,821, titled “Balloon Tipped Pad Delivery Catheter” and filed on Mar. 31, 2022, the disclosure of which is hereby incorporated by reference herein.

### BACKGROUND OF THE DISCLOSURE

Valvular heart disease, and specifically aortic and mitral valve disease, is a significant health issue in the United States. Traditional valve replacement surgery, the orthotopic replacement of a heart valve, is an “open heart” surgical procedure. Briefly, the procedure necessitates a surgical opening of the thorax, initiation of extra-corporeal circulation with a heart-lung machine, stopping and opening the heart, excision and replacement of the diseased valve, and re-starting of the heart. While valve replacement surgery typically carries a 1-4% mortality risk in otherwise healthy persons, a significantly higher morbidity is associated with the procedure, largely due to the necessity for extra-corporeal circulation. Further, open heart surgery is often poorly tolerated in elderly patients. Thus, if the extra-corporeal component of the procedure could be eliminated, morbidities and cost of valve replacement therapies would be significantly reduced.

While replacement of the aortic valve in a transcatheter manner is the subject of intense investigation, lesser attention has traditionally been focused on the mitral valve. This is in part reflective of the greater level of complexity associated with the native mitral valve and thus a greater level of difficulty with regard to inserting and anchoring the replacement prosthesis.

Recent developments in the field have provided devices and methods for mitral valve replacement with reduced invasion and risk to the patient. Such devices may include a prosthetic valve disposed within the native valve annulus and held in place, at least in part, with an anchor pad seated against an exterior surface of the heart near the apex. In some instances, the anchor pad may be inserted with a catheter navigated transseptally through the myocardium of the ventricle and through a puncture made in the ventricular wall, which can create several potential issues. For example, blood within the ventricle may be susceptible to leaking through the puncture formed in the ventricular wall prior to deployment of the anchor pad. Further, the space between the ribs and the exterior of the heart can be very small, such as about 0.5 inches (12.6 mm) or less, requiring great precision during deployment of the anchor pad. Still further, transseptal mitral valve replacement is typically performed as a beating heart procedure (i.e. without stopping the heart and placing the patient on cardiopulmonary bypass). The beating of the heart during the procedure can increase the difficulty of maintaining stability and control of the pad delivery catheter while attempting to deploy the anchor. In particular, it may be difficult to maintain the distal tip of the catheter at the desired location relative to the puncture in the heart wall due to the beating movement of the heart. Accordingly, methods and devices for anchoring a prosthetic heart valve that address one or more of these issues may be desirable.

### BRIEF SUMMARY OF THE DISCLOSURE

According to a first aspect of the disclosure, a method for delivering an anchor to a surface of a heart may comprise intravascularly navigating a catheter to a wall of the heart; passing the catheter through a puncture in the wall of the heart; inflating a balloon coupled to a distal end of the catheter, the balloon positioned radially outward of the distal end of the catheter; translating an anchor disposed within the catheter in a distal direction relative to the catheter to deploy the anchor from the distal end of the catheter; deflating the balloon; and retracting the catheter proximally to remove the catheter from the heart.

According to another aspect of the disclosure, a prosthetic heart valve delivery system may include a prosthetic heart valve, an anchor and a catheter. The catheter may extend from a proximal end to a distal end. The catheter may be configured to receive the prosthetic heart valve and the anchor in collapsed conditions within the catheter. The catheter may include a balloon and an inflation lumen. The balloon may be positioned radially outward of the catheter at the distal end of the catheter. The inflation lumen may extend through the catheter from the proximal end to the distal end. The inflation lumen may be in fluid communication with the balloon.

### BRIEF DESCRIPTION OF THE DRAWINGS

FIG. 1 is a longitudinal cross-section of an anchor delivery catheter according to an embodiment of the disclosure.

FIG. 2A is a transverse cross-section of the anchor delivery catheter of FIG. 1.

FIG. 2B is an end view of an anchor delivery catheter according to another embodiment of the disclosure.

FIG. 3 is a perspective view of an anchor configured to be delivered by the anchor delivery catheter of FIG. 1.

FIG. 4 is an axial view of the anchor of FIG. 3.

FIG. 4A is a side view of an anchor according to another embodiment of the disclosure.

FIG. 5 illustrates a trans-jugular insertion of a delivery catheter for the anchor of FIG. 3.

FIG. 6 illustrates a trans-femoral insertion of the delivery catheter of FIG. 3.

FIG. 7 is a schematic view of the pad delivery catheter of FIG. 1 extending through a ventricular wall of a heart.

FIGS. 8-10 illustrate the anchor of FIG. 3 in progressive stages of deployment from the delivery catheter of FIG. 1.

FIG. 11 illustrates a prosthetic heart valve implanted and anchored in a heart.

### DETAILED DESCRIPTION

As used herein, the term “proximal,” when used in connection with a delivery device or components of a delivery device, refers to the end of the device closer to the user of the device when the device is being used as intended. On the other hand, the term “distal,” when used in connection with a delivery device or components of a delivery device, refers to the end of the device farther away from the user when the device is being used as intended. As used herein, the terms “substantially,” “generally,” “approximately,” and “about” are intended to mean that slight deviations from absolute are included within the scope of the term so modified. Unless otherwise stated, like reference numerals refer to like elements throughout the disclosure.

The present application describes an anchor delivery catheter which may be used to deliver an epicardial pad or anchor to an apex of a heart. After the anchor is deployed from the catheter and placed in abutment with an exterior surface of the ventricular wall of the heart, the anchor may be coupled to a prosthetic mitral valve (e.g. via a tether) positioned within the native mitral valve to securely hold the prosthetic mitral valve in place while the prosthetic valve performs the general function of a healthy native mitral valve.

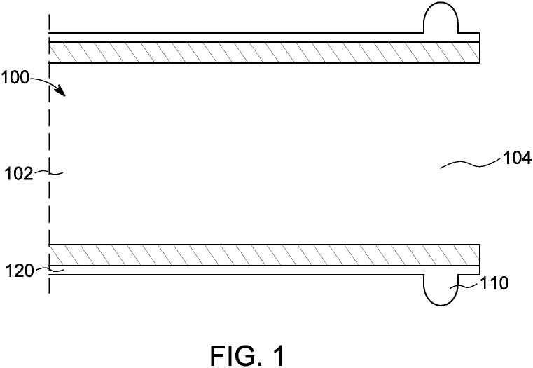

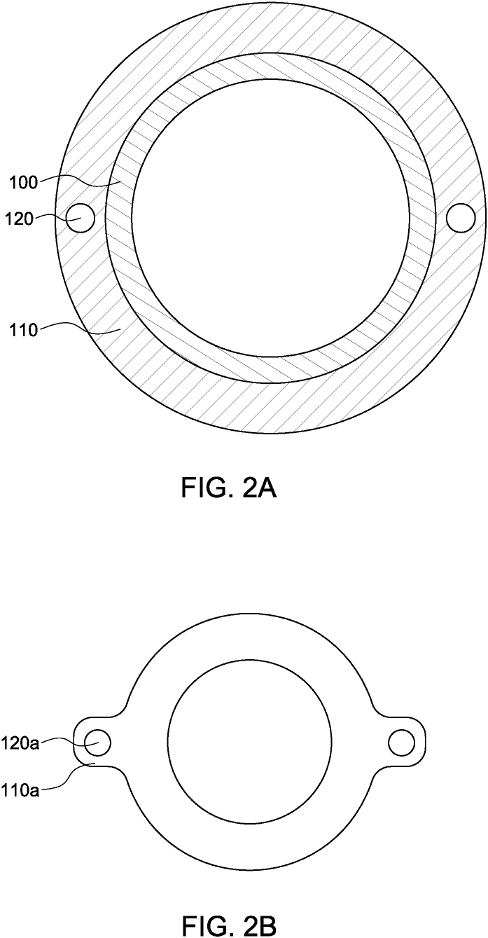

FIGS. 1 and 2A illustrate an anchor delivery catheter 100 extending from a proximal end 102 to a distal end 104. It should be noted that delivery catheter 100 may extend farther in the proximal direction than what is shown in FIG. 1, however, for ease of description, proximal end 102 refers to the true proximal-most end of the delivery catheter. Delivery catheter 100 may have a length sufficient to extend through several passageways of a body and heart of a patient, as described below in greater detail, while still being accessible to a surgeon or operator outside of the patient’s body at its true proximal end. Delivery catheter 100 may have an elongate cylindrical shape with a generally circular cross section along a plane perpendicular to the proximal-distal direction. It is also contemplated that the delivery catheter may have any other cross-sectional shape, such as an oval, a stadium-shape, or a triangle or rectangle with rounded edges.

A balloon 110 or balloon ring is coupled at distal end 104 of delivery catheter 100 extending circumferentially around an outer surface of the distal end of the delivery catheter. Balloon 110 is inflatable and adapted to transition between an inflated configuration and a deflated configuration. While in the deflated configuration, balloon 110 may be substantially flush against the outer surface of delivery catheter 100, only marginally increasing the diameter of the delivery catheter by the thickness of the balloon material. As such, delivery catheter 100 may be navigated through the body with a minimal diameter by maintaining balloon 110 in the deflated configuration until the balloon reaches its destination. Balloon 110 is shown in FIGS. 1 and 2A in the inflated configuration. As shown in FIG. 1, balloon 110 extends radially outward from delivery catheter 100 while in the inflated configuration, but preferably only a small axial distance, e.g., less than about 0.5 inches (1.27 cm) in some embodiments and less than about 0.25 inches (0.635 cm) in other embodiments. As shown in FIG. 2A, balloon 110 preferably expands uniformly around the full circumference of distal end 104. In some examples, balloon 110 may be formed of a compliant material such that the balloon is able to expand non-uniformly. That is, if a portion of balloon 110 abuts a surrounding object while the balloon is being inflated, the balloon may be configured to substantially stop expanding where the portion contacts the surrounding object and may be configured to continue inflating around the remaining portions of the balloon not contacting a surrounding object.

An inflation lumen 120 extends proximally from balloon 110 along the length of delivery catheter 100. That is, inflation lumen 120 extends from proximal end 102, where it is in communication with a fluid reservoir such as a syringe filled with saline, to distal end 104 where it is in fluid communication with the interior volume of balloon 110. The inflation lumen 120 may extend along the outer surface of delivery catheter 100 (e.g., the inflation lumen is positioned radially outward of the delivery catheter) and in communication with balloon 110. Inflation lumen 120 is accessible to the operator at proximal end 102 for the operator to inject a substance (e.g., a saline solution, carbon dioxide, or the like) through the inflation lumen and into balloon 110 to inflate the balloon. Inflation lumen 120 may be surrounded by balloon 110 such that the inflation lumen does not protrude radially outward from an outer surface of the balloon, as shown in FIG. 2A. In some embodiments, the inflation lumen 120 may be positioned within a wall of delivery catheter 100, with an opening at the distal end of the catheter 100 opening into the balloon 110. In other examples, inflation lumen 120a may protrude radially outward from the outer diameter of delivery catheter 100, as shown in FIG. 2B which may increase the amount of space defined internally within delivery catheter 100, e.g., increasing the available space for other components within the internal diameter, to prevent interference of the inflation lumen with the anchor 210 disposed therein. Delivery catheter 100 may be rigidly constructed such that the pathway through the delivery catheter defining inflation lumen remains generally undeformed during manipulation of the delivery catheter and inflation/deflation of balloon 110. It is contemplated that a delivery catheter may include more than one inflation lumen, and each inflation lumen may be circumferentially spaced along the outer surface of the delivery catheter extending from the proximal end to the distal end. In the illustrated example, two inflation lumens 120 are shown spaced about 180 degrees apart from each other.

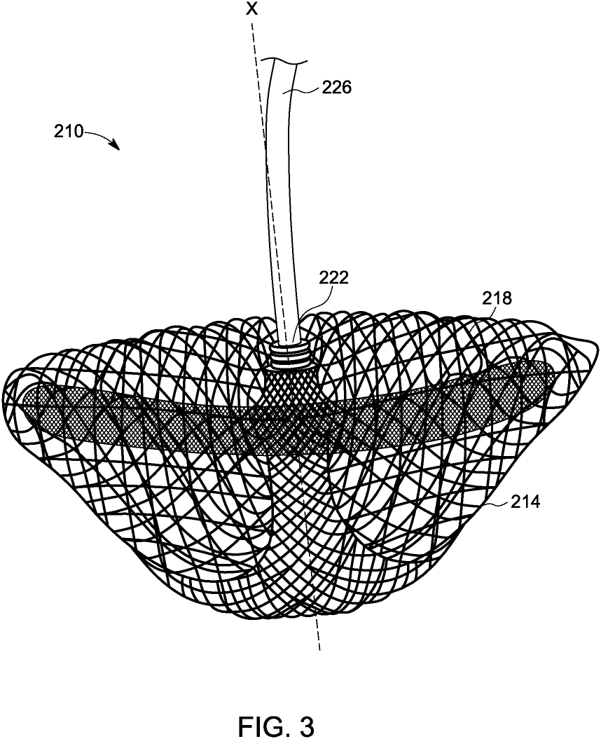

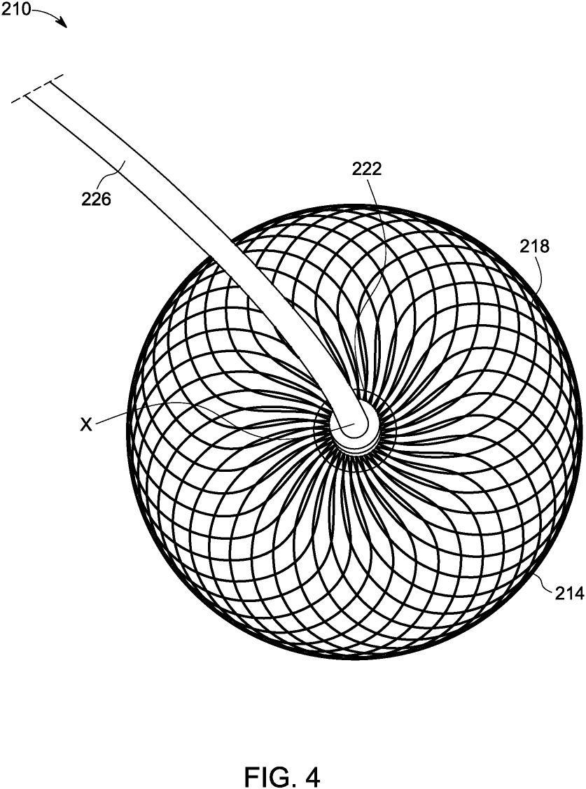

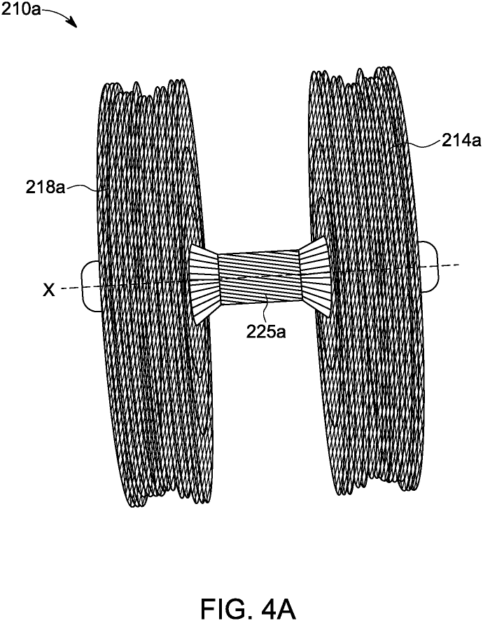

An exemplary anchor 210 for a prosthetic mitral heart valve is illustrated in FIGS. 3 and 4 and described in greater detail in U.S. Provisional Pat. Application 63/195,770, the disclosure of which is fully incorporated by reference herein. Anchor 210 includes a first disc 214 and may optionally include a second disc 218, both provided by a wire mesh and centered on an axis X. First disc 214 is offset from second disc 218 in a first direction along axis X. First disc 214 and second disc 218 are each biased toward a dome-shaped resting configuration that is concave toward a second direction along axis X, the second direction being opposite the first direction. The resting configuration of first disc 214 extends far enough in the second direction along axis X to partially overlap second disc 218.

It should be understood that the illustrated dome shapes are merely exemplary, and first disc 214 and second disc 218 may be biased differently. For example, either or both of first disc 214 and second disc 218 may be biased toward a resting configuration that is convex toward the second direction or generally planar. Further, the first disc 214 and second disc 218 may be biased to different resting configurations. In one example, the first disc 214 may be biased toward a dome-shaped resting configuration that is concave toward the second direction while the second disc 218 is biased toward a generally planar configuration having about the same diameter location as the widest part of the dome-shaped resting configuration of the first disc 214. In further examples, the first disc 214 may be concave toward the second direction while the second disc 218 is concave toward the first direction such that the concave portions of the first and second disc face each other. In still further examples, an anchor 210a may have a first disc 214a and a second disc 218a connected to each other by a neck 225a, wherein each of the first and second discs are generally shaped like wheels in the expanded configuration as shown in FIG. 4A.

Anchor 210 also includes a cuff, anchor cap 222, or other connector for holding the braids of the anchor together and/or for gripping a tether 226, which may be connected to a prosthetic heart valve. It is also contemplated that tether 226 may extend through anchor cap 222 and couple and/or anchor to a distal portion of the braids. Anchor cap 222 is offset from second disc 218 in the second direction along axis X. One-way gripping features, such as angled teeth, within anchor cap 222 may permit anchor 210 to slide along tether 226 in the second direction, but not the first direction. Again, it is noted that the anchor 224 is merely exemplary, and any type of anchor may be disposed within and deployed from delivery catheter 100 as described below in greater detail.

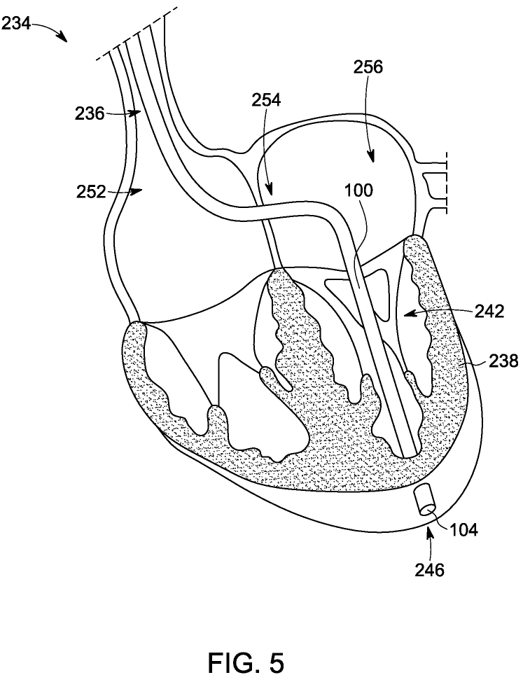

FIG. 5 shows a trans-jugular insertion of an at least partially flexible delivery catheter 100 for the delivery of anchor 210. Delivery catheter 100 may be formed of any known material for building catheters, including biocompatible metals such as steel, polymers, etc., and may be part of a steerable or flexible catheter system. Delivery catheter 100 may include a relatively inflexible portion near its distal end to facilitate the intended puncture of tissue and guidance of anchor 210. Delivery catheter 100 is inserted through the patient’s jugular vein (not shown), then through superior vena cava 236, right atrium 252, atrial septum 254, left atrium 256, native mitral valve 260, and into left ventricle 242. Delivery catheter 100 exits left ventricle 242 through ventricular wall 238 at or near the apex 246 of heart 234. A retractable puncturing device (not shown) and a retractable atraumatic tip (not shown) may extend from the distal open end 104 of delivery catheter 100 in alternate stages of insertion of the delivery catheter. The puncturing device may produce openings through atrial septum 254 and ventricular wall 238 while the atraumatic tip may act to prevent injury to other tissue. In some embodiments, separate devices may be used to create the puncture in the atrial septum and the puncture at the ventricular apex. Once delivery catheter 100 has been delivered to its destination, the distal open end 104 of delivery catheter is positioned outside of ventricular wall 238. The trans-jugular insertion of delivery catheter 100 may be accomplished by any of variety of methods, such as, for example, guiding the delivery catheter along a guide wire, such as a shape-memory guide wire, inserted through the jugular vein. The flexible nature of anchor 210 allows trans-jugular delivery of anchor 210 through delivery catheter. Because delivery catheter 100 and anchor 210 reach heart 234 from the jugular vein, the anchor may be delivered and implanted without any intercostal puncture.

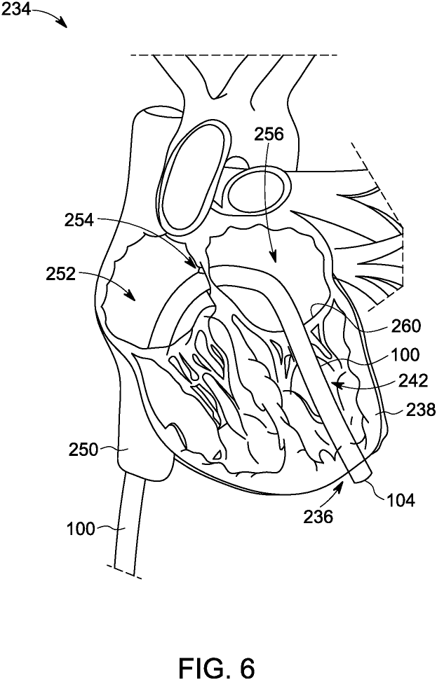

FIG. 6 shows a trans-femoral insertion of delivery catheter 100. Delivery catheter 100 is introduced into the patient via the femoral vein, enters heart 234 through inferior vena cava 250, travels through right atrium 252, and punctures septum 254 to enter left atrium 256. Delivery catheter 100 is advanced from left atrium 256 through native mitral valve 260, left ventricle 242, and ventricular wall 238 such that open distal end 104 of the delivery catheter is positioned outside of ventricular wall 238 at or near apex 246. As with trans-jugular insertion, guidance of delivery catheter 100 during trans-femoral insertion may be accomplished using a variety of methods, including guidance along a guide wire.

The trans-jugular and trans-femoral insertions described above are merely exemplary. It should be understood that delivery catheter 100 could be guided toward heart 234 using any suitable method known in the art. It should be understood that, although not show, an atraumatic tip may be provided at the distal end of the delivery catheter 100 (e.g. a separate atraumatic balloon that may be inflated to create the atraumatic distal tip, and deflated to allow for devices to pass through the distal end of the catheter 100.

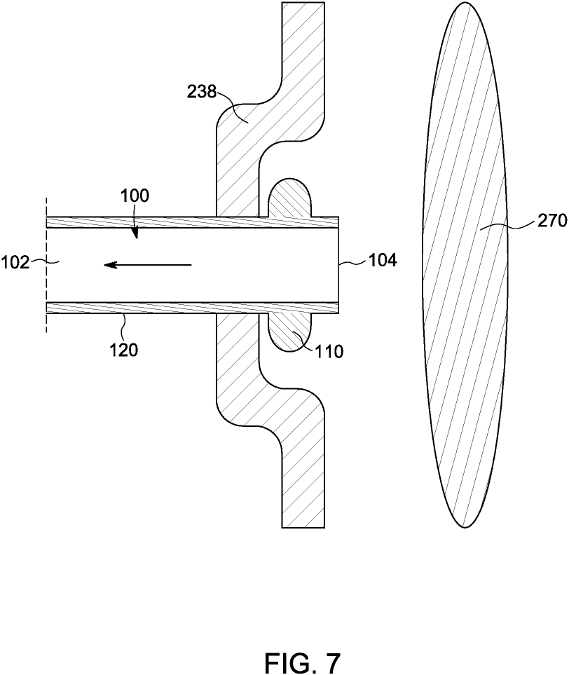

FIG. 7 illustrates delivery catheter 100 extending through ventricular wall 238 of heart 234, with the left side of the view of FIG. 7 representing the inside of the heart and the right side of the view of FIG. 7 representing outside the heart. Distal end 104 of delivery catheter 100 is passed from an inner side of ventricular wall 238 to an outer side of the ventricular wall through a puncture in the wall. As noted above, the puncture may be made via a separate needle that has already been withdrawn in the view of FIG. 7, and an atraumatic tip may have been removed (e.g. deflated and pulled proximally through the delivery catheter 100) after the distal end of the delivery catheter 100 crosses the ventricular wall via the puncture. After distal end 104 of delivery catheter 100 is passed through ventricular wall 238, balloon 110 may be inflated via inflation lumen 120 so that the balloon expands radially outward. By expanding balloon 110 radially outward, the diameter of the balloon becomes greater than the diameter of the puncture through ventricular wall 238, which prevents delivery catheter 100 from being withdrawn or retracted back into left ventricle 242 through the ventricular wall while the balloon 110 remains inflated. Delivery catheter 100 may be retracted proximally to abut a generally proximal-facing surface of balloon 110 against ventricular wall 238, and the delivery catheter may be further retracted proximally to apply pressure between the balloon and the ventricular wall, and may deform the ventricular wall in the proximal direction. Applying proximal pressure and maintaining significant contact between balloon 110 and ventricular wall 238 may prevent leakage of blood out of the left ventricle 242 through the space between the outer diameter of the delivery catheter 100 and the puncture of the ventricular wall. Further, deformation of ventricular wall 238 in the proximal direction may provide additional space between the ventricular wall and the rib bones 270 to allow for an easier and improved deployment of anchor 210 from delivery catheter 100. In other words, by pulling the heart tissue proximally, additional space is created in which the anchor pad may self-expand and be deployed. Still further, the pressure between balloon 110 and ventricular wall 238 may help stabilize delivery catheter 100 while anchor 210 is being deployed, which might otherwise be susceptible to shaking or agitation caused by the actively beating heart 234. In other words, the position of the terminal distal end of the catheter relative to the heart tissue adjacent the puncture remains substantially fixed due to the contact and pressure between the balloon 110 and the heart tissue, even though the heart is beating. It is also contemplated that, in some embodiments, while balloon 110 is in the deflated configuration, it may be positioned substantially within the puncture of the ventricular wall 238, i.e., between the inner and outer sides of the ventricular wall, and may be inflated in this position to create a form fit with the balloon to close the puncture while anchor 210 is deployed.

In some examples, balloon 110 may be provided with a drug coating that is configured to be transferred from the balloon to surrounding tissue upon contact with the balloon. For instance, balloon 110 may be inflated on the outer side of ventricular wall 238 (or within the puncture) and while being retracted proximally to abut the outer surface of the ventricular wall, drug particles coating the balloon such as an anti-inflammatory drug may be transferred to the tissue of heart 234 to reduce swelling which may have been caused by the puncture or the navigation of delivery catheter 100.

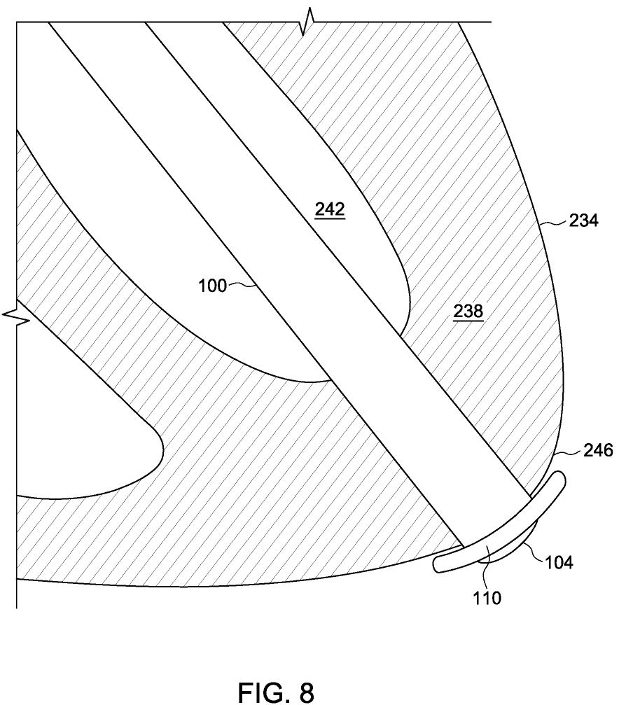

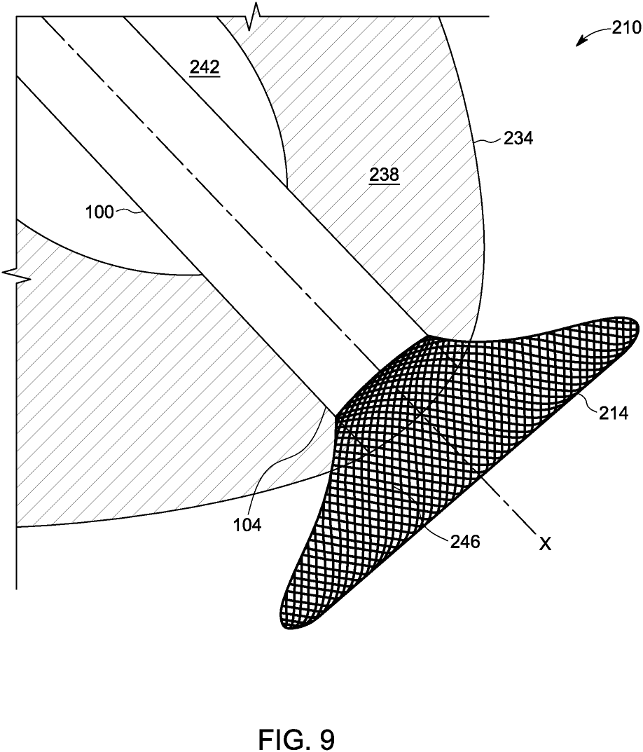

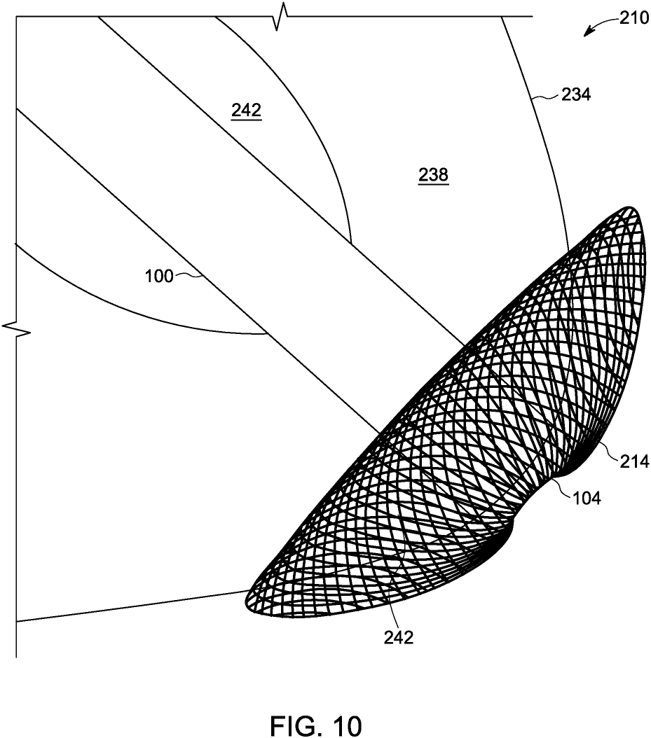

FIGS. 8-10 illustrate anchor 210 in progressive stages of deployment from the open distal end 104 of delivery catheter 100. Deployment of anchor 210 from delivery catheter 100 is shown without a balloon surrounding the delivery catheter in FIGS. 9 and 10, but it is noted that deployment may be executed in substantially the same manner as described below but with a balloon surrounding the delivery catheter as shown in FIGS. 7 and 8. Delivery catheter 100 is shown in a distalmost position in FIG. 8, with open distal end 104 positioned outside of heart 234. Anchor 210 may be translated distally relative to delivery catheter 100 to deploy the anchor. For example, a semi-rigid cable or wire (not shown) may be inserted through delivery catheter 100 to contact the proximal end of anchor 210. Translating the wire distally to push anchor 210 distally relative to delivery catheter 100 causes the anchor to deploy out from open distal end 104 of the delivery catheter. In some examples, delivery catheter 100 may include a prosthetic heart valve disposed within the delivery catheter proximal to anchor 210, such that the semi-rigid cable wire contacts the prosthetic heart valve which pushes the anchor out distal end 104. As shown in FIG. 9, pushing anchor 210 distally relative to delivery catheter 100 causes first disc 214 of the anchor to deploy out from the open distal end 104 of the delivery catheter and expand radially relative to axis X. Upon further advancement of the anchor 210, the bias of first disc 214 causes it to curve back onto the outer apex 246 of heart 234, as shown in FIG. 10. It should be noted that as described above, delivery catheter 100 shown in FIG. 9 would include inflated balloon 110 circumferentially surrounding distal end 104, and instead of the delivery catheter 100 being retracted back through the puncture of ventricular wall 238, the retraction of the delivery catheter will cause the balloon to press against the outer surface of the ventricular wall and possibly deform the wall slightly in the proximal direction. In some examples in which anchor 210 includes second disc 218 or 218a, further advancement of the anchor in combination with deflating the balloon and retracting delivery catheter 100 in the proximal direction may allow second disc 218 or 218a to deploy and expand radially relative to axis X within left ventricle 242 until second disc 218 or 218a opens to press against an inner side of wall 238. Pressure against the outer surface of ventricular wall 238 may result from applying tension to tether 226 in some examples or the elastic bias of first disc 214 or 214a and second disc 218 or 218a toward certain resting positions in other examples.

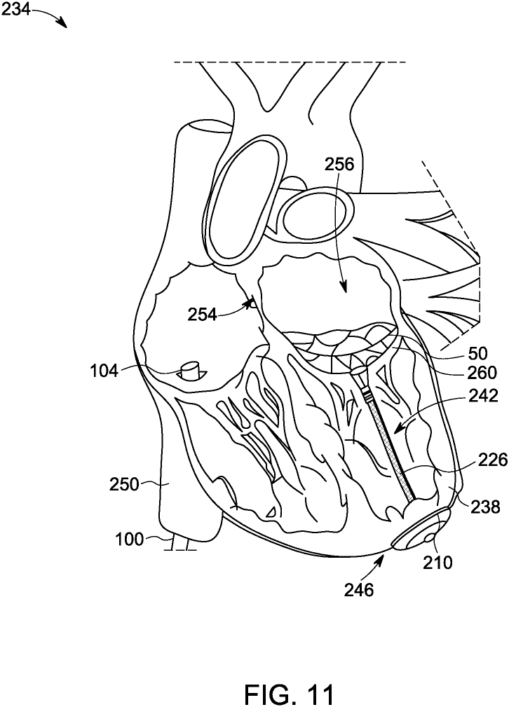

In some examples, after anchor 210 is fully deployed and balloon 110 has been deflated, delivery catheter 100 may be retracted proximally from left ventricle 242 such that distal end 104 is substantially positioned within or near native mitral valve 260. With prosthetic heart valve 50 tethered to anchor 210 and still disposed within delivery catheter 100, the prosthetic heart valve may be deployed from distal end 104 of delivery catheter in a substantially similar manner, e.g., retracting the delivery catheter while applying distal pressure to the prosthetic heart valve with the semi-rigid cable. Prosthetic heart valve 50 may then be positioned and desirably placed within native mitral valve 260. FIG. 11 illustrates prosthetic heart valve 50 implanted in heart 234 with anchor 210 seated at or near the apex 246 of heart 234. Delivery catheter 100 has been withdrawn from heart 234, through inferior vena cava 250 in the illustrated example, leaving valve 50 behind.

According to one aspect of the disclosure, a method for delivering an anchor to a surface of a heart comprises:
intravascularly navigating a catheter to a wall of the heart;
passing the catheter through a puncture in the wall of the heart;
inflating a balloon coupled to a distal end of the catheter, the balloon positioned radially outward of the distal end of catheter;
translating an anchor disposed within the catheter in a distal direction relative to the catheter to deploy the anchor from the distal end of the catheter;
deflating the balloon; and
retracting the catheter proximally to remove the catheter from the heart; and/or
navigating the catheter includes passing the catheter through an atrial septum of the heart into a left atrium, and passing the delivery catheter through a native mitral valve into a left ventricle toward an inner surface of the wall of the heart; and/or
passing the delivery catheter through the wall of the heart includes creating the puncture in a ventricular wall of the heart, and passing the delivery catheter from the left ventricle through the puncture in the ventricular wall to extend outside of the heart; and/or
inflating the balloon includes injecting a saline solution into the balloon through an inflation lumen extending along the catheter and in communication with the balloon at the distal end of the catheter; and/or
deflating the balloon includes withdrawing a saline solution from the balloon through an inflation lumen extending along the catheter and in communication with the balloon at the distal end of the catheter; and/or
translating the catheter in a proximal direction while the balloon is inflated to abut the balloon against an outer surface of the wall of the heart; and/or
translating the catheter in the proximal direction causes the wall of the heart to deform in the proximal direction by applying pressure from the balloon on the outer surface of the wall in the proximal direction; and/or
while the balloon is inflated, the balloon has a diameter greater than a diameter of the puncture in the wall of the heart through which the catheter is passed; and/or
when passing the delivery catheter through the wall of the heart, the catheter passes from an inner side of the wall to an outer side of the wall; and/or
when the balloon is inflated, the balloon is positioned on the outer side of the wall; and/or
when the balloon is inflated, the balloon is positioned between the inner side of the wall and the outer side of the wall; and/or
an outer surface of the balloon is drug-coated, and the method further comprising contacting surrounding tissue with the outer surface of the balloon to transfer the drug from the balloon to the surrounding tissue; and/or
deploying a prosthetic heart valve tethered to the anchor in a native mitral valve after the step of deflating the balloon; and/or
the balloon circumferentially surrounds the distal end of the catheter.

According to another aspect of the disclosure, a prosthetic heart valve delivery system comprises:
a prosthetic heart valve;
an anchor; and
a catheter extending from a proximal end to a distal end and configured to receive the prosthetic heart valve and the anchor in collapsed conditions within the catheter, the catheter comprising:
   a balloon positioned radially outward of the catheter at the distal end of the catheter; and
   an inflation lumen extending through the catheter from the proximal end to the distal end, the inflation lumen in fluid communication with the balloon; and/or
the balloon is configured to inflate uniformly to define a substantially uniform outer diameter in an inflated configuration; and/or
the inflatable balloon is formed of a compliant material such that when a first portion of the balloon contacts a surrounding object, the first portion of the balloon is configured to stop expanding and a second portion of the balloon is configured to continue expanding while the balloon is inflated; and/or
the inflation lumen includes a plurality of inflation lumens spaced circumferentially around the catheter and extending from the proximal end to the distal end in communication with the balloon; and/or
in an inflated configuration, the inflation lumen and balloon form a substantially uniform outer diameter around the catheter; and/or
the inflation lumen protrudes radially outward relative to an outer diameter of the catheter; and/or
a drug coating an outer surface of the balloon, the drug configured to be transferred to a surrounding medium when contacted by the balloon; and/or
a tether having a first end and a second end, wherein the prosthetic heart valve is configured to receive the first end of the tether and the anchor is configured to receive the second end of the tether.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method for delivering an anchor to a surface of a heart comprising:
intravascularly navigating a catheter to a wall of the heart;
passing the catheter through a puncture in the wall of the heart;
inflating a balloon coupled to a distal end of the catheter, the balloon positioned radially outward of the distal end of the catheter;
translating an anchor disposed within the catheter in a distal direction relative to the catheter to deploy the anchor from the distal end of the catheter;
deflating the balloon; and
retracting the catheter proximally to remove the catheter from the heart.

2. The method of claim 1, wherein navigating the catheter includes passing the catheter through an atrial septum of the heart into a left atrium, and passing the delivery catheter through a native mitral valve into a left ventricle toward an inner surface of the wall of the heart.

3. The method of claim 1, wherein passing the delivery catheter through the wall of the heart includes creating the puncture in a ventricular wall of the heart, and passing the delivery catheter from the left ventricle through the puncture in the ventricular wall to extend outside of the heart.

4. The method of claim 1, wherein inflating the balloon includes injecting a saline solution into the balloon through an inflation lumen extending along the catheter and in communication with the balloon at the distal end of the catheter.

5. The method of claim 1, wherein deflating the balloon includes withdrawing a saline solution from the balloon through an inflation lumen extending along the catheter and in communication with the balloon at the distal end of the catheter.

6. The method of claim 1, further comprising translating the catheter in a proximal direction while the balloon is inflated to abut the balloon against an outer surface of the wall of the heart.

7. The method of claim 6, wherein translating the catheter in the proximal direction causes the wall of the heart to deform in the proximal direction by applying pressure from the balloon on the outer surface of the wall in the proximal direction.

8. The method of claim 1, wherein while the balloon is inflated, the balloon has a diameter greater than a diameter of the puncture in the wall of the heart through which the catheter is passed.

9. The method of claim 1, wherein when passing the delivery catheter through the wall of the heart, the catheter passes from an inner side of the wall to an outer side of the wall.

10. The method of claim 9, wherein when the balloon is inflated, the balloon is positioned on the outer side of the wall.

11. The method of claim 9, wherein when the balloon is inflated, the balloon is positioned between the inner side of the wall and the outer side of the wall.

12. The method of claim 1, wherein an outer surface of the balloon is drug-coated, and the method further comprising contacting surrounding tissue with the outer surface of the balloon to transfer the drug from the balloon to the surrounding tissue.

13. The method of claim 1, further comprising deploying a prosthetic heart valve tethered to the anchor in a native mitral valve after the step of deflating the balloon.

14. A prosthetic heart valve delivery system, comprising:
a prosthetic heart valve;
an anchor; and
a catheter extending from a proximal end to a distal end and configured to receive the prosthetic heart valve and the anchor in collapsed conditions within the catheter, the catheter comprising:
a balloon positioned radially outward of the catheter at the distal end of the catheter; and
an inflation lumen extending through the catheter from the proximal end to the distal end, the inflation lumen in fluid communication with the balloon.

15. The system of claim 14, wherein the balloon is configured to inflate uniformly to define a substantially uniform outer diameter in an inflated configuration.

16. The system of claim 14, wherein the inflatable balloon is formed of a compliant material such that when a first portion of the balloon contacts a surrounding object, the first portion of the balloon is configured to stop expanding and a second portion of the balloon is configured to continue expanding while the balloon is inflated.

17. The system of claim 14, wherein the inflation lumen includes a plurality of inflation lumens spaced circumferentially around the catheter and extending from the proximal end to the distal end in communication with the balloon.

18. The system of claim 14, wherein in an inflated configuration, the inflation lumen and balloon form a substantially uniform outer diameter around the catheter.

19. The system of claim 14, wherein the inflation lumen protrudes radially outward relative to an outer diameter of the catheter.

20. The system of claim 14, further comprising a drug coating an outer surface of the balloon, the drug configured to be transferred to a surrounding medium when contacted by the balloon.

21. The system of claim 14, further comprising a tether having a first end and a second end, wherein the prosthetic heart valve is configured to receive the first end of the tether and the anchor is configured to receive the second end of the tether.
